(19) United States
(12) Patent Application Publication
(10) Pub. No.: US 2008/0269913 A1
(43) Pub. Date: Oct. 30, 2008

(54) ARTIFICIAL LEG

(75) Inventors: Dieter Gobbers, Heidenheim (DE); Walter Gobbers, Heidenheim (DE)

Correspondence Address: BAKER & DANIELS LLP;111 E. WAYNE STREET, SUITE 800, FORT WAYNE, IN, 46802, US

(73) Assignee: REGO FLORIDA CORPORATION, Lehigh Acres, FL (US)

(21) Appl. No.: 11/740,615

(22) Filed: Apr. 26, 2007

(51) Int. Cl. A61F 2/60; A61F 2/74

(52) U.S. Cl. 623/27; 623/33

(57) ABSTRACT

The invention concerns an artificial leg with an artificial knee and an artificial foot. The artificial leg according to the invention comprises the following features:
the artificial knee has a joint body turnable about its turning axis for carrying a femoral stump;
with a shaft, the upper end of which engages the joint body, and the lower end of which supports the artificial foot;
the artificial foot is connected to the shaft in a telescopically expandable manner;
there is provided a conrod drive, comprising a connecting rod, the lower end of which is lead by the shaft in a slideable manner, and engages the artificial foot;
the upper end of the connecting rod is articulated to a lever arm, which is in driving connection with the joint body, so that during the bending of the artificial knee the distance between its turning axis and the tip of the artificial foot is reduced.

The invention concerns an artificial leg with an artificial knee and an artificial foot. Such a prosthesis can be attached to a leg stump above the knee.

Numerous embodiments have become known. FR 2 711 512 A1 describes a prosthesis, by means of which the patient can perform a swinging movement. The swinging movement is controlled, so that a comparatively natural walking can be obtained.

Numerous documents address the control of the relative movement of the upper part of an artificial leg, which means the part above the joint prosthesis, facing the lower part, which means the part below the joint prosthesis. In this context, in many cases the damping of the advance is concerned. See GB-A-2 252 503, WO 93/22 991 or WO 92/22 267.

Problems occur during the so called swinging phase, which means that phase, during which, when striding, the patient pivots the artificial leg in walking direction. In this, a streaking contact between the artificial foot and the floor occurs. This leads to an unsecure walking of the patient.

The invention is based on the task to design an artificial leg of the said kind in such a manner that the walking of the patient is improved, especially in the swinging-out phase.

This problem is solved by the features of claim 1.

Accordingly, during the bending of the knee joint, the distance between its joint axis and the tip of the artificial foot is reduced. The artificial leg as a whole is hereby reduced during bending. Thereby, a streaking of the artificial foot or its tip at the floor is avoided. It is further possible to lift only the foot of the artificial leg or the foot's tip. In this context, a further joint is necessary, either between the foot of the artificial leg and the main part of the artificial leg, or the foot's tip and the main part of the foot of the artificial leg.

The invention is further explained with reference to the drawing(s). In detail, therein is shown the following:

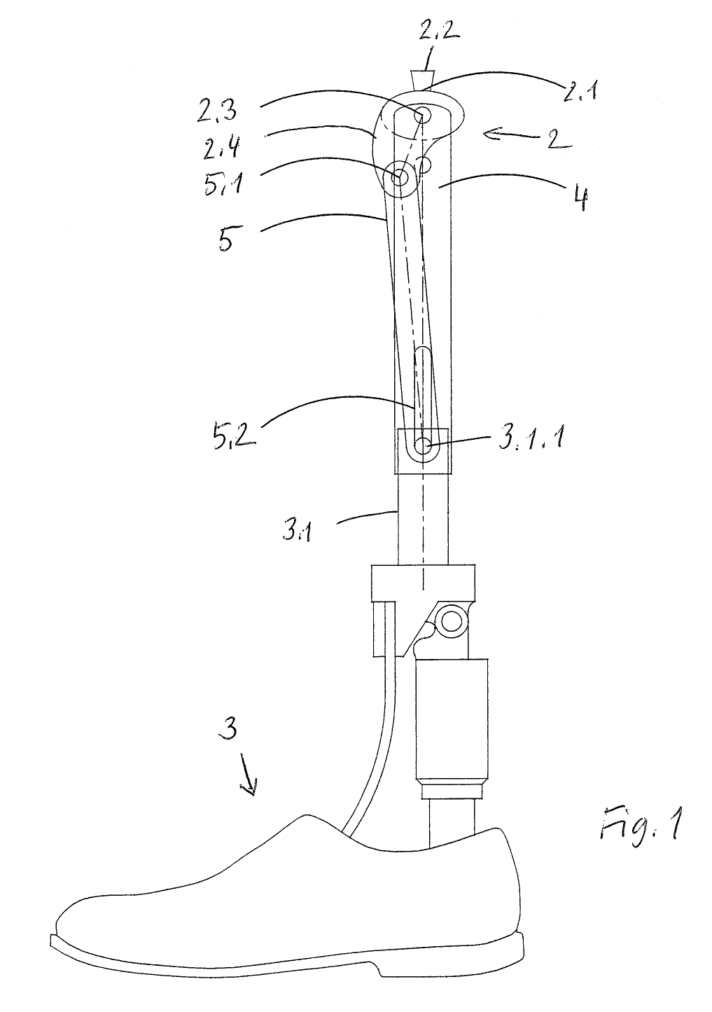

FIG. 1 shows a side view of an artificial leg according to a first embodiment at a first bending angle of the artificial knee.

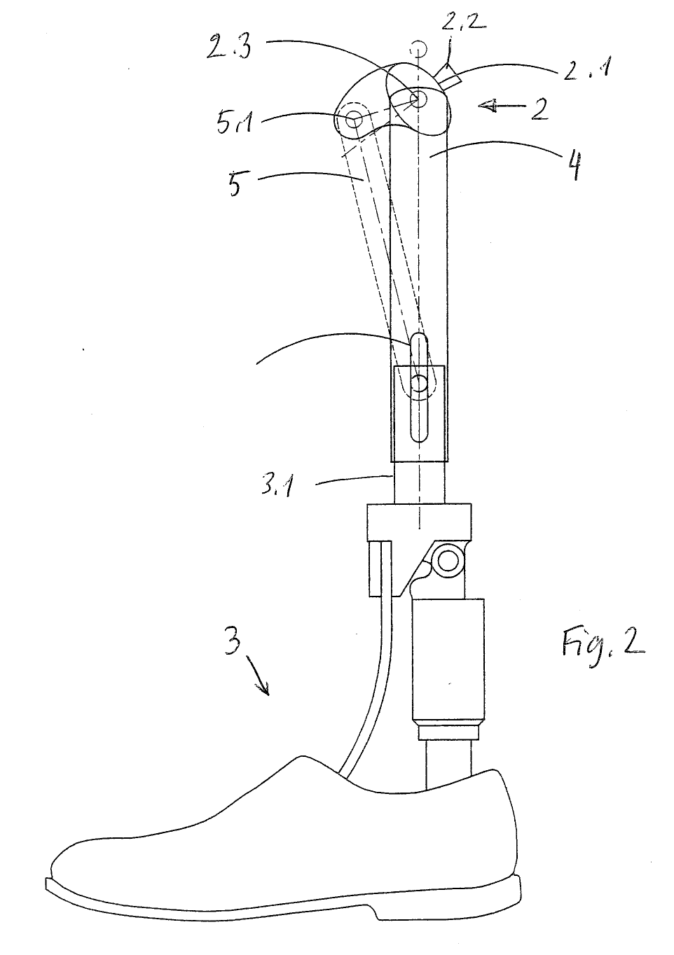

FIG. 2 shows the subject of FIG. 1 in a second bending position of the artificial knee.

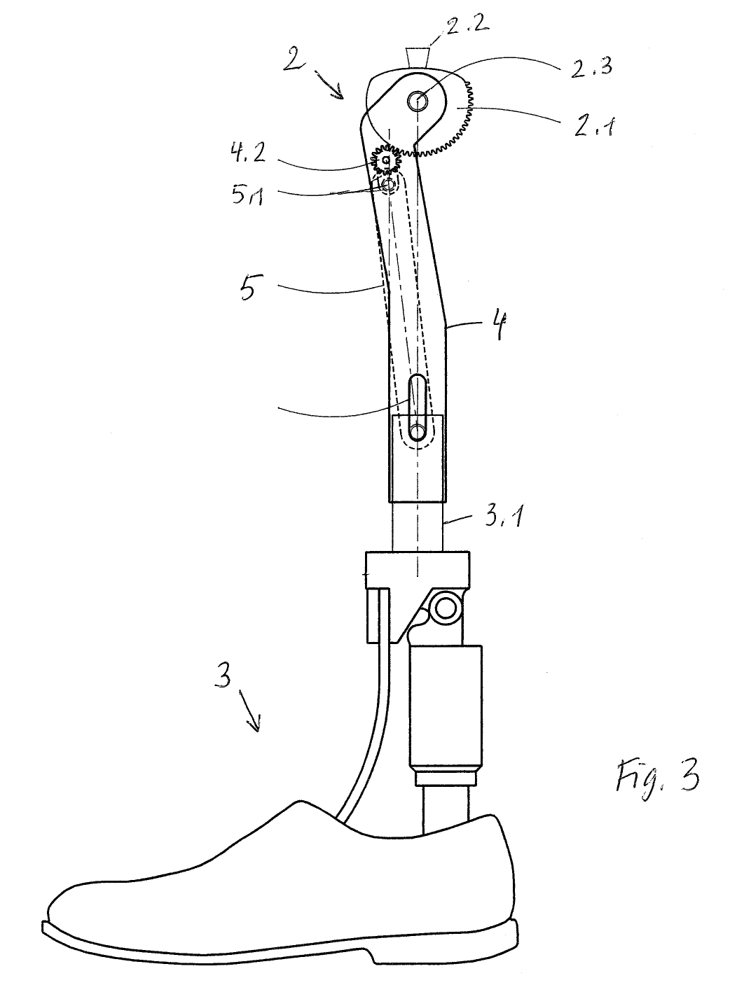

FIG. 3 shows in side view a second embodiment of the invention in a first bending position of the artificial knee.

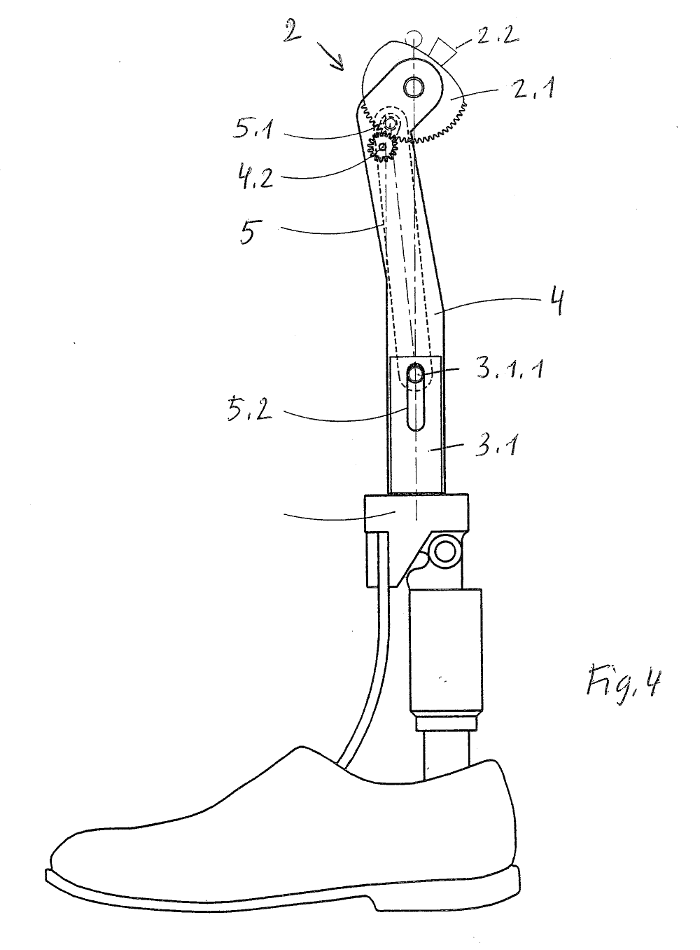

FIG. 4 shows the subject of FIG. 3 in a second bending position of the artificial knee.

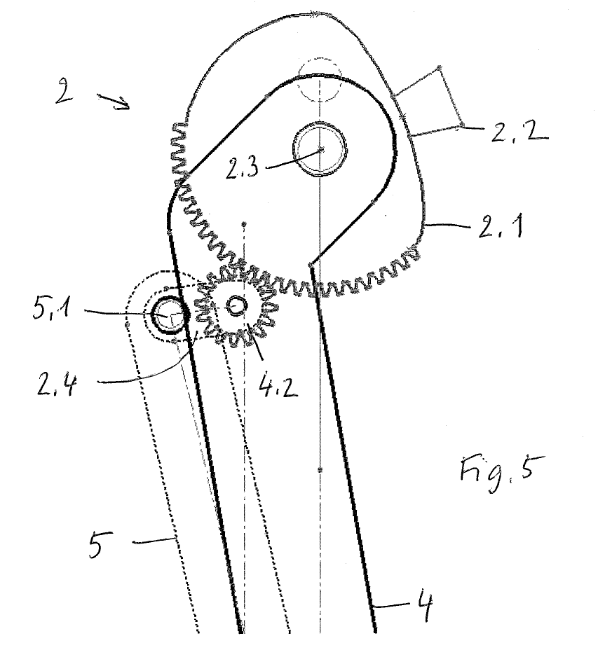

FIG. 5 shows the upper part of the subject of FIGS. 3 and 4 in enlarged view.

The artificial leg 1 according to the FIGS. 1 to 4 comprises besides others an artificial knee 2 and an artificial foot 3.

In FIGS. 1 and 2, according to the first embodiment of the invention, a shaft 4 is to be seen. This carries the artificial knee 2 at its upper end. This comprises a joint body 2.1, (and) further a connection sleeve for a receiving cup (here not shown) for receiving a femoral stump.

A lever arm 2.4 is molded to the joint body 2.1. At the free end of the lever arm 2.4, a connecting rod 5 is articulated. The lower end of the connecting rod in turn engages an attachment pin 3.1 of the artificial foot 3 in an articulating manner. The attachment pin is enclosed by the shaft 4, and can slide upwards and downwards in it. See the elongated hole 5.2 in the connecting rod 5, as well as the bolt 3.1.1 of the attachment pins 3.1 engaging it.

When bending the artificial knee 2, the joint body 2.1 is twisted about the turning axis 2.3 of the joint. The turning point 5.1 of the connecting rod 5 moves in a circular arc about the turning axis 2.3 of the joint. Thereby, the free end of the connecting rod 5 will be moved upwards, and thereby the artificial foot 3 is lifted as well.

From the comparison of the FIGS. 1 and 2, it is to be noted that a twist of the joint body 2.1 has lead to a lifting of the artificial foot 3. See also the angular position of the connection sleeve 2.2 of the joint body 2.1.

In the second embodiment according to the FIGS. 3, 4 and 5, a conrod drive is again provided. The connection between the shaft 4 and the artificial foot 3 is therefore generally the same as in case of the first embodiment according to the FIGS. 1 and 2.

The joint body 2.1 is provided with a toothing at its outer periphery though. Further, a pinion 4.2 is provided. This is supported at the shaft 4 in a turnable manner. It meshes with the toothing of the joint body 2.1. It is again to be seen the connecting rod 5, the lower end of which engages the attachment pin 3.1 of the artificial foot 3.

It is provided a lever arm 2.4. This is, to the one hand, articulated to the upper end the connecting rod 5, and to the other hand to the pinion 4.2. This can be seen especially well in FIG. 5, especially the lever arm 2.4.

In this embodiment, a bending of the artificial knee also leads to a lifting of the artificial foot, and thereby de facto to a shortening of the artificial leg as a whole.

It can as well be sufficient, not only to lift the acute portion of the artificial foot 3, but instead the whole artificial foot.

In case of the second embodiment according to the FIGS. 3, 4 and 5, the shaft 4 is cranked at two positions. The reasons for this are related to production as well as to mounting.

Generally, the shaft can be of tube form.

In the seated position of the patient—with a bending angle of about 90°—the artificial leg is again elongated, in order to obtain a knee height, which is equal to that of the healthy leg.

In the present embodiment, the lower end of the connecting rod 5 is mounted in a slideable manner by means of the shaft 4. Instead of this, a lever mechanics, for example redirections, might be provided, by help of which the lower end of the connecting rod 5 is connected to the artificial foot 3.

### LIST OF REFERENCE SIGNS

1 artificial leg
2 artificial knee
2.1 joint body
2.2 connection sleeve
2.3 turning axis of the joint
2.4 lever arm
3 artificial foot
3.1 attachment pin
3.1.1 bolt
4 shaft
4.2 pinion
5 connecting rod
5.1 turning point
5.2 elongated hole

1. Artificial leg with an artificial knee and an artificial foot;
the artificial knee having, for carrying a femoral stump, a joint body, which is turnable about its turning axis;
with a shaft, the upper end of which is engaged at the joint body, and the lower end of which bears the artificial foot;
wherein the artificial foot is connected to the shaft in a telescopically expandable manner,
wherein there is provided a conrod drive comprising a connecting rod, the lower end of which is engaged at the artificial foot;
wherein the upper end of the connecting rod is articulated to a lever arm, which is in driving connection to the joint body, so that during the bending of the artificial knee the distance between its turning axis and the tip of the artificial foot is reduced.

2. Artificial leg according to claim 1, characterized in that the lever arm is unitary with the joint body.

3. Artificial leg according to claim 1, characterized by the following features:
a pinion is supported at the shaft;
the lever arm is articulated at the upper end of the connecting rod as well as at the pinion;
the joint body has inner or outer teeth, and meshes with the pinion.

4. Artificial leg according to claim 1, characterized in that the lower end of the connecting rod by means of a lever mechanics, for example redirections, engages the artificial foot.

6. Artificial leg according to claim 2, characterized in that the lower end of the connecting rod by means of a lever mechanics, for example redirections, engages the artificial foot.

7. Artificial leg according to claim 3, characterized in that the lower end of the connecting rod by means of a lever mechanics, for example redirections, engages the artificial foot.
